SEQUENCE LISTING (12 sequences)

SEQ ID NO: 1 - DNA, 664 bp, Zea mays
  CDS (1)..(501)
ggcgggggttacggcggtgcaaccggcaaggcttcctcaggcggcggcggactggaccccgacggcgacccagaggttgggctgaacgggaaggcgatcgaggagatcgtgaacgagcacaacgtgttccgcgccaaggagcacgtgcctccgctggtgtggaacgcgacgctggccaggttctcgcagcagtacgcggagacgctcaagggcaactgccagcagatccactcgtcgtcgccgtacggggagaacctgatggagggcaccccggggctgacctggaatatcaccgtggacggctggagcgaggagaagaagaactaccactacgactcggacacctgcgaccccggcaagatgtgcggccactacaaggccgtcgtctggaagaccaccaccagcgtcggctgcggacgcatcaagtgcaacagcggcgacaccatcatcatgtgcagctactggccgccggggaactaccacggcgttaagccatactaaaatatatatcgacccattgcatatatatatgttgcagagctttttattttggcaacatatctagtgcatatggactgctggtatacatcatgcacatattaaatcctatcctttaatttaagatccattgtcttacaattgaaaaaaaaaaaaaaaaaaaaaa

SEQ ID NO: 2 - protein, 166 aa, Zea mays
GGGYGGATGKASSGGGGLDPDGDPEVGLNGKAIEEIVNEHNVFRAKEHVPPLVWNATLARFSQQYAETLKGNCQQIHSSSPYGENLMEGTPGLTWNITVDGWSEEKKNYHYDSDTCDPGKMCGHYKAVVWKTTTSVGCGRIKCNSGDTIIMCSYWPPGNYHGVKPY

SEQ ID NO: 3 - DNA, 772 bp, Zea mays
  CDS (56)..(571)
ctaaacatcacagtattacacactgacagatcaacgacaataagctttgtcagcaatggaggtagtctcgtcgaagctagcttggttcgcgctcgcgttcaccgtggtggccgccgccgctgccggccgtgtcgtctcggcccagaacacggcgcaggacttcgtgaacctgcacaactccccgcgcgcggacgtgggcgtcgggaacgtggcctggaacaccacggtggcggcgtacgcgcagagctacgcgaaccagcgcgcgggcgactgccggctggtgcactccggcgggccctacggggagaacctgttctggggctcggcgggctacgcctggacggcgtcgaacgccgtgggatcctgggcggcggagaagcagtactacaaccacgccaccaacacctgctccgctccgtccggccagtcgtgcggccactacacgcagctggtgtggcgcgcctccactgccatcggatgcgcccgcgtcgtctgcagcaacaacgccggcgtcttcatcatctgcaactattacccgccgggcaacgtgattggacagagcccttactagcaacctctatatatatacgtacttgcaataatccgcgctatatccacaagtgtgtgtaacctactaacctacatacatttgtgagcagcagcatcaatcatacgcgcgtgtgatgccatgcatgccagcatgagttgttacatatgttcgccccgcatatgcatttcaagaattaattttttaaaaaaaaaaaaaaaaaaa

SEQ ID NO: 4 - protein, 171 aa, Zea mays
MEVVSSKLAWFALAFTVVAAAAAGRVVSAQNTAQDFVNLHNSPRADVGVGNVAWNTTVAAYAQSYANQRAGDCRLVHSGGPYGENLFWGSAGYAWTASNAVGSWAAEKQYYNHATNTCSAPSGQSCGHYTQLVWRASTAIGCARVVCSNNAGVFIICNYYPPGNVIGQSPY

SEQ ID NO: 5 - DNA, 906 bp, Zea mays
  CDS (46)..(618)
gccaaggagaaggcgtacgcggcggagaaggtcgtccagcaggagatgctcaagtacgccaaggagaagggtctggtgtccccgaccaacggcacggggtggtacaggggcatcgcccgggagttcgtggacgcccacaacgagctccgcgcgcgctacggcgtgccgcccatgaagtgggacaaccagctggcgcggcaggcgcggcgctggtccaacgccatgcgcaaggactgccagatcctccacagcggccacgagtacggcgagagcgtgttcaggagctacgacgactggaacgccaccgccagggaggccgtcttctggtggggcaaggaggaggccatctacgacaaggacaaggagaagtgcaagtacggcaaggtcttcaaggagtgcggccacttcgcgctcatggtcggcaagaggagcaccaaggtcggcttgcgcacgagccgagtgctttcaagggcggcgtcttcatcacctgcaactactatgcaacggacctggacaagaacaagaacaaaacaccaaacgataactgactctatccacccatctgtttgttcctatgtacgtacgtacgtagtagcgctgacttttttactgaggtgatgattggattgttaattaactactactgaagcaattgcctgattccgaggttggagatgatgacgatgaagacgacgactactacgtacgagaggatcctcctccctcttcttcttttttgttttttaattttgttcttccaattttgggcccaatcagtcaagcagattgtatctatatctatatatatacacacgatcgggtacagcagacagccaattgatcagttcatttcagttgggaaaaaaaaaaaaaaaaaaaaaaaaaaaaaaaaaaaaaaaaaaaaaa

SEQ ID NO: 6 - protein, 190 aa, Zea mays
MLKYAKEKGLVSPTNGTGWYRGIAREFVDAHNELRARYGVPPMKWDNQLARQARRWSNAMRKDCQILHSGHEYGESVFRSYDDWNATAREAVFWWGKEEAIYDKDKEKCKYGKVFKECGHFALMVGKRSTKVGLRTSRVLSRAASSSPATTMQRTWTRTRTKHQTITDSIHPSVCSYVRTYVVALTFLLR

SEQ ID NO: 7 - DNA, 395 bp, Zea mays
  CDS (3)..(197)
cggaggcccagtggtacgactacggcagcaactcctgcgccgcgccgccgggcgccgggtgcctccggtacacgcaggtcgtctggcgcaacaccacgcagctcggctgcgcccggatcgtgtgcgactccggcgatactttcctggcttgcgactacttcccgccaggcaactacggcacgggcaggccctactgaccgtacgtcgtcagttgatgctgtcatctgcgggccgggccgcgcacacattggtttcccatgcagtactctgcgttgaattggtgtcctttgaattgcatatgtcgttccgtttcctctgtcgattcttcaggctgctgtttcgccggtacgctaaaaataaaacgaaaaatttagtaaaaaaaaaaaaaaaaaaaa

SEQ ID NO: 8 - protein, 64 aa, Zea mays
EAQWYDYGSNSCAAPPGAGCLRYTQVVWRNTTQLGCARIVCDSGDTFLACDYFPPGNYGTGRPY

SEQ ID NO: 9 - DNA, 525 bp, Zea mays
  CDS (1)..(285)
ttccccgacggccagttcgcgctcggggagaacgtcttctggggcgggcccggcggcgcgtggcggccccgggacgccgtcgcggactgggccgccgagggcgccgactactcgtacgccgacaacgcgtgcgcgccaggccgggagtgcgcgcactacacccagatcgtgtggcgacgcaccaccgccgtcggctgcgcccgggtggcgtgcgacggcggcggggtgttcatcacctgcaactactacccgcccggcaacgtcgtcggcgagaggccgtactaactagctagcttagcaactcagtaccctcggcctcggggatttattacgtgacgtggcgaacggcctcgtgtagccgtgtcgtgtgtaaataatattcagcggtcgttccggtgaacatgatggtaattcactcgagtttttcactgttggcttccgtaaatcgtgtttggtttgaccagaggcctcagagccacgatcacaataaaatgtggttttacaaaaaaaaaaaaaaaaaaaaaa

SEQ ID NO: 10 - protein, 94 aa, Zea mays
FPDGQFALGENVFWGGPGGAWRPRDAVADWAAEGADYSYADNACAPGRECAHYTQIVWRRTTAVGCARVACDGGGVFITCNYYPPGNVVGERPY

SEQ ID NO: 11 - DNA, 364 bp, Zea mays
  Unsure (345)..(345): “n” at position 345  can be a, c, g, or t.
cagccgtcgccgcctggctctccgagcgcccccgctacgactactggaccaacagctgctacggcggctccatgtgcggccactacacccagatcatgtggccggacacccgacgcgtcggatgcgccatggtcgcttgctacggcggcaggggcaccttcatcacctgcaactacgacccgccgggcaactacgtcggcctgcgcccgtactgacaccgttcctttcgtcgtctggccgccgccgccggccagtgtctcttgctgtcccaaatcgttgctgctacggtgctaccactatgatatcgagatgacatgacaatgtgtacaaatgttgcctggctancgttattatacttgctnat

SEQ ID NO: 12 - protein, 70 aa, Zea mays
AVAAWLSERPRYDYWTNSCYGGSMCGHYTQIMWPDTRRVGCAMVACYGGRGTFITCNYDPPGNYVGLRPY

(19) United States
(12) Patent Application Publication
(10) Pub. No.: US 2002/0166146 A1
(43) Pub. Date: Nov. 7, 2002

(54) Maize PR1 polynucleotides and methods of use

(75) Inventors: Carl R. Simmons, Des Moines, IA (US); Pedro A. Navarro Acevedo, Ames, IA (US); Virginia C. Crane, Des Moines, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/068,347

(22) Filed: Feb. 6, 2002

Related U.S. Application Data
(60) Provisional application No. 60/267,052, filed on Feb. 7, 2001.

(51) Int. Cl. A01H 1/00; C07H 21/04; C12N 5/04

(52) U.S. Cl. 800/288; 435/417; 435/320.1; 536/23.2

(57) ABSTRACT

The present invention provides compositions and methods for enhancing the disease resistance of plants. The compositions are novel nucleic acid molecules isolated from maize, which encode pathogenesis-related (PR1) proteins, and the PR1 proteins encoded thereby. The methods comprise introducing into a plant cell at least one nucleotide construct comprising a PR1 nucleotide sequence of the invention operably linked to a promoter that drives expression in a plant cell. The methods additionally involve regenerating a stably transformed plant from the transformed plant cell. Transformed plants and seeds having enhanced disease resistance are also provided.

### CROSS-REFERENCE TO RELATED APPLICATIONS

This application claims the benefit of U.S. Provisional Application No. 60/267,052, filed Feb. 7, 2001, which is hereby incorporated herein in its entirety by reference.

### FIELD OF THE INVENTION

The present invention relates to the field of plant molecular biology, more particularly to regulation of gene expression and enhancing disease resistance in plants.

### BACKGROUND OF THE INVENTION

Disease in plants is caused by biotic and abiotic causes. Biotic causes include fungi, viruses, bacteria, and nematodes. Of these, fungi are the most frequent causative agent of disease on plants. Abiotic causes of disease in plants include extremes of temperature, water, oxygen, soil pH, plus nutrient-element deficiencies and imbalances, excess heavy metals, and air pollution.

A host of cellular processes enables plants to defend themselves from disease caused by pathogenic agents. These processes apparently form an integrated set of resistance mechanisms that is activated by initial infection and then limits further spread of the invading pathogenic microorganism.

Subsequent to recognition of a potentially pathogenic microbe, plants can activate an array of biochemical responses. Generally, the plant responds by inducing several local responses in the cells immediately surrounding the infection site. The most common resistance response observed in both nonhost and race-specific interactions is termed the “hypersensitive response” (HR). In the hypersensitive response, cells contacted by the pathogen, and often neighboring cells, rapidly collapse and dry in a necrotic fleck. Other responses include the deposition of callose, the physical thickening of cell walls by lignification, and the synthesis of various antibiotic small molecules and proteins, among which are the pathogenesis-related (PR) proteins. Genetic factors in both the host and the pathogen determine the specificity of these local responses, which can be very effective in limiting the spread of infection.

Pathogenesis-related proteins, which have been described in a number of plants (see Bowles (1990) Ann. Rev. Biochem. 59:873-907 for review), include the PR1 proteins. Although their biochemical functions remain unknown, expression of PR1 proteins is generally induced by pathogens and many abiotic treatments associated with the elicitation of the defense response, more particularly a hypersensitive response (see WO 89/02437 for a review). In tobacco, PR1 protein expression is induced by viral infection and salicylic acid (SA) treatment (van Loon et al. (1987) Plant Mol. Biol. 9:593; Ward et al (1991) Plant Cell 3:1085). Barley plants resistant to powdery mildew caused by Erysiphe graminis accumulate PRb-1 (a basic PR1) mRNA 12 hours after inoculation with that pathogen, while susceptible plants do not, indicating these proteins serve as antipathogenic agents that contribute to disease resistance. Ethylene, jasmonic acid (JA), and SA also induce the accumulation of PRb-1 in the resistant cultivars, but not in related susceptible lines (Muradov et al. (1993) Plant Mol. Biol. 23:439). Salicylic acid induces PR1 protein accumulation in maize leaves. Ultraviolet light and C. carbonum (tox-) inoculations induce protein accumulation in Pr (hm1) leaves (in Crane et al. (1996), Biology of plant-Microbe Interactions (International Society for Molecular Plant-Microbe Interactions), pp. 223-226). These observations make maize PR1 nucleotide sequences and their promoters ideal candidates for use in the development of transgenic plants, particularly transgenic plants having enhanced disease resistance.

Thus, isolation and characterization of PR1 nucleotide sequences and their corresponding promoters, which can serve as regulatory regions for expression of their native gene or other heterologous nucleotide sequences of interest, are needed for genetic manipulation of plants to exhibit specific phenotypic traits, particularly enhanced disease resistance, either in response to a given stimulus or in a constitutive manner.

### BRIEF SUMMARY OF THE INVENTION

Compositions of the invention include isolated nucleotide sequences for novel maize PR1 nucleotide sequences and isolated pathogenesis proteins encoded thereby. The PR1 nucleotide sequences and proteins find use in enhancing the disease resistance of plants. The PR1 nucleotide sequences are useful in the genetic manipulation of any plant or plant cell for increased resistance to pathogens. To express a PR1 nucleotide sequence of the invention in a plant or plant cell, the plant or plant cell can be transformed with a nucleotide construct comprising a PR1 nucleotide sequence of the invention operably linked to a promoter that drives gene expression in a plant cell. In this manner, transformed plants and progeny thereof having increased resistance to pathogens and their related diseases can be obtained. Thus, the invention further provides plant cells, plant tissues, plants and seeds thereof transformed with the PR1 nucleotide sequences of the invention.

### DETAILED DESCRIPTION OF THE INVENTION

Compositions of the present invention are polynucleotides, or nucleic acid molecules, comprising novel PR1 nucleotide sequences and respective predicted amino acid sequences which encode pathogenesis-related class I (PR1) proteins. The maize PR1 nucleotide sequences, designated as PR1-6, PR1-7, PR1-8, PR1-9, PR1-10, and PR1-11, are set forth in SEQ ID NOS: 1, 3, 5, 7, 9, and 11, respectively. Amino acid sequences for the PR1 proteins encoded by these genes are set forth in SEQ ID NOS: 2, 4, 6, 8, 10, and 12, respectively.

In particular, the present invention provides for isolated nucleic acid molecules comprising nucleotide sequences encoding the amino acid sequences shown SEQ ID NOS: 2, 4, 6, 8, 10, and 12. Further provided are polypeptides having an amino acid sequence encoded by a nucleic acid molecule described herein, for example those set forth in SEQ ID NOS: 1, 3, 5, 7, 9, and 11.

Thus, the invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid molecule is free of sequences (preferably protein encoding sequences) that naturally flank the nucleic acid molecule (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid molecule is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably, culture medium represents less than about 30%, 20%, 10%, or 5% (by dry weight) of chemical precursors or non-protein-of-interest chemicals.

Compositions of the invention include the nucleotide sequences for six maize PR1 nucleotide sequences as set forth in SEQ ID NOS: 1, 3, 5, 7, 9, and 11 and the corresponding amino acid sequences for the PR1 proteins encoded thereby as set forth in SEQ ID NOS: 2, 4, 6, 8, 10, and 12, respectively. These gene sequences may be assembled into a nucleotide construct such that the gene is operably linked to a promoter that drives expression of a coding sequence in a plant cell. Plants stably transformed with this nucleotide construct express, either in a constitutive or inducible manner, a PR1 protein of the invention. Expression of this protein creates or enhances disease resistance in the transformed plant.

Fragments and variants of these native nucleotide and amino acid sequences are also encompassed by the present invention. By “fragment” is intended a portion of the nucleotide or amino acid sequence. Thus, for example, less than the entire promoter sequences disclosed herein may be utilized to drive expression of an operably linked nucleotide sequence of interest, such as a nucleotide sequence encoding a heterologous protein. It is within skill in the art to determine whether such fragments decrease expression levels or alter the nature of expression, i.e., constitutive or inducible expression. Alternatively, fragments of a promoter nucleotide sequence that are useful as hybridization probes, such as described below, generally do not retain this regulatory activity.

Fragments of a PR1 nucleotide sequence of the invention may encode protein fragments that retain the biological activity of the native PR1 protein, i.e., the sequences set forth in SEQ ID NOS: 2, 4, 6, 8, 10, and 12, and hence enhance disease resistance when expressed in a plant. Alternatively, fragments of a coding nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the entire nucleotide sequence encoding the proteins of the invention.

A fragment of a PR1 nucleotide sequence that encodes a biologically active portion of a PR1 protein of the invention will encode at least 15, 25, 30, 40, 50, 75, 100, or 150 contiguous amino acids, or up to the total number of amino acids present in a full-length PR1 protein of the invention (for example, 166, 171, 190, 64, 94, or 70 amino acids for SEQ ID NOS: 2, 4, 6, 8, 10, or 12, respectively). Fragments of a PR1 nucleotide sequence that are useful as hybridization probes for PCR primers generally need not encode a biologically active portion of a PR1 protein.

A biologically active portion of a PR1 protein can be prepared by isolating a portion of one of the PR1 nucleotide sequences of the invention, expressing the encoded portion of the PR1 protein (e.g., by recombinant expression in vitro), and assessing the activity of the encoded portion of the PR1 protein. Nucleic acid molecules that are fragments of a PR1 nucleotide sequence comprise at least 15, 20, 50, 75, 100, 325, 350, 375, 400, 425, 450, 500, 550, 600, 650, 700, or 800 nucleotides, or up to the number of nucleotides present in a full-length PR1 nucleotide sequence disclosed herein (for example, 664, 772, 906, 395, 525, or 364 nucleotides for SEQ ID NO: 1, 3, 5, 7, 9, or 11, respectively).

By “variants” is intended sequences having substantial similarity with a promoter or gene nucleotide sequence disclosed herein. For nucleotide sequences, conservative variants include those sequences that, because of the degeneracy of the genetic code, encode the amino acid sequence of one of the PR1 proteins of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode a PR1 protein of the invention. Generally, variants of a particular nucleotide sequence of the invention will have at least about 40%, 45%, 50%, 55%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, antipathogenic activity or disease resistance as described herein. Such variants may result from, for example, genetic polymorphism or from human manipulation. Biologically active variants of a native PR1 protein of the invention will have at least about 40%, 45%, 50%, 55%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the PR1 proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra (eds.) Techniques in Molecular Biology, MacMillan Publishing Company, NY (1983) and the references cited therein.

Thus, the PR1 nucleotide sequences of the invention include both the naturally occurring sequences as well as mutant forms. Likewise, the proteins of the invention encompass both naturally occurring proteins as well as variations and modified forms thereof Such variants will continue to possess the desired PR1 antipathogenic defense protein activity. Obviously, the mutations that will be made in the DNA encoding a variant protein must not place the sequence out of reading frame and preferably will not create complementary regions that could produce secondary mRNA structure. See, for example, EP Patent Application Publication No. 75,444.

In this manner, the present invention encompasses the PR1 proteins as well as components and fragments thereof. That is, it is recognized that component polypeptides or fragments of the proteins may be produced which retain PR1 protein activity that enhances disease resistance in a plant. These fragments include truncated sequences, as well as N-terminal, C-terminal, internal and internally deleted amino acid sequences of the proteins.

The deletions, insertions, and substitutions of the protein sequences encompassed herein are not expected to produce radical changes in the characteristics of the PR1 proteins. However, when it is difficult to predict the exact effect of the substitution, deletion, or insertion in advance of doing so, one skilled in the art will appreciate that the effect will be evaluated by routine screening assays. That is, the activity of the modified protein sequences can be evaluated by monitoring of the plant defense system. See, for example U.S. Pat. No. 5,614,395, herein incorporated by reference.

Variant nucleotide sequences and proteins also encompass sequences and proteins derived from a mutagenic and recombinogenic procedure such as DNA shuffling. With such a procedure, one or more different PR1 nucleotide sequences can be manipulated to create a new PR1 nucleotide sequence which encodes s new PR1 protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between two or more PR1 nucleotide sequences of the invention to obtain a new gene coding for a protein with an improved property of interest, such as increased disease resistance, a broadened pathogen specificity, or both. Alternatively, using this approach, sequence motifs encoding a domain of interest may be shuffled between one or more PR1 nucleotide sequences of the invention and other known PR1 genes to obtain a new gene coding for a protein with an improved property of interest. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Thus the PR1 nucleotide sequences of the present invention include the native forms as well as fragments and variants thereof. Similarly, the PR1 proteins of the invention include the native forms as well as fragments and variants thereof. The variant nucleotide sequences and variant proteins will share substantial homology with their naturally occurring sequences. By “substantial homology” is intended a sequence exhibiting substantial functional and structural equivalence with the native or naturally occurring sequence. Any functional or structural differences between substantially homologous sequences do not effect the ability of the sequence to function as a PR1 protein as disclosed in the present invention. Two nucleotide sequences or polypeptides are considered substantially homologous when they have at least about 40%, 45%, 50%, 55%, 60%, 65%, to 70%, generally at least about 80%, preferably at least about 85%, 90%, to 98% sequence homology. Substantially homologous sequences of the present invention include variants of the disclosed sequences such as those that result from site-directed mutagenesis, as well as synthetically derived sequences.

The nucleotide sequences of the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to an entire PR1 sequence set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the PR1 sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire PR1 sequence disclosed herein, or one or more portions thereof, may be used as a probe capable of specifically hybridizing to corresponding PR1 sequences and messenger RNAs. To achieve specific hybridization under a variety of conditions, such probes include sequences that are unique among PR1 sequences and are preferably at least about 10 nucleotides in length, and most preferably at least about 20 nucleotides in length. Such probes may be used to amplify corresponding PR1 sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode a PR1 protein and which hybridize under stringent conditions to at least one of the PR1 sequences disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang etal. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. to about 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent wash conditions are those in which the salt concentration is about 0.02 molar at pH 7 and the temperature is at least about 50, 55, or 60° C. However, nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al. (1970) J. Mol. BioL 48:443. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The invention involves transforming host cells with the nucleotide constructs of the invention. Generally, the nucleotide construct will comprise a PR1 nucleotide sequence of the invention operably linked to a promoter that drives expression in the host cell of interest. In this manner, the PR1 proteins of the invention can be produced in any host cell of interest. Host cells include, but are not limited to: plant cells; animal cells; fungal cells, particularly yeast cells; and bacterial cells. Methods for transforming host cells and expressing proteins therein are known in the art. Promoters that drive gene expression in a host cell of interest are also known in the art.

The PR1 nucleotide sequences and proteins described herein may be used alone or in combination with other PR1 nucleotide sequences or proteins, or any other nucleotide sequences, proteins or agents, to protect against plant diseases and pathogens. Other plant defense proteins and nucleotide sequences include, but are not limited to, those described in the copending applications both entitled “Methods for Enhancing Disease Resistance in Plants”, U.S. application Ser. No. 60/076,151, filed Feb. 26, 1998, and U.S. application Ser. No. 60/092,464, filed Jul. 11, 1998, and WO 99/43819; all of which are herein incorporated by reference.

The PR1 nucleotide sequences disclosed herein are useful for genetic engineering of plants to express a phenotype of interest. The PR1 nucleotide sequences, when operably linked with a promoter that drives expression in a plant cell, may be used to create or enhance disease resistance in a transformed plant.

Where the promoter and its native gene are naturally occurring within a plant, i.e., in maize, transformation of the plant with these operably linked sequences results in a change in phenotype, such as enhanced disease resistance, or insertion of these operably linked sequences within a different region of the chromosomes thereby altering the plant's genome.

Thus, in one embodiment of the invention, expression cassettes will comprise a transcriptional initiation region, or promoter, operably linked to a PR1 nucleotide sequence of the invention. Such an expression cassette is provided with a plurality of restriction sites for insertion of the nucleotide sequence to be under the transcriptional regulation of the regulatory regions. The expression cassette may additionally contain selectable marker genes.

The transcriptional cassette will include in the 5′-to-3′ direction of transcription, a transcriptional and translational initiation region, a heterologous nucleotide sequence of interest, and a transcriptional and translational termination region functional in plants. The termination region may be native with the transcriptional initiation region comprising one of the promoter nucleotide sequences of the present invention, may be native with the DNA sequence of interest, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; Joshi et al. (1987) Nucleic Acid Res. 15:9627-9639.

The PR1 nucleotide sequences of the invention are provided in expression cassettes for expression in the plant of interest. The cassette will include 5′ and 3′ regulatory sequences operably linked to the gene of interest. The expression cassette may also contain at least one additional nucleotide sequence for a gene to be cotransformed into the organism. Alternatively, the additional sequence(s) can be provided on another expression cassette.

Where appropriate, the PR1 nucleotide sequences of the invention may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, 5,436,391, and Murray et al. (1989) Nucleic Acids Res. 1 7:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al (1987) Plant Physiol. 84:965-968. Other methods known to enhance translation can also be utilized, for example, introns, and the like.

In those instances where it is desirable to have the expressed product of the PR1 nucleotide sequence directed to a particular organelle, such as the chloroplast or mitochondrion, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase, and the like.

The nucleotide sequences of the present invention can also be used to suppress the expression of endogenous PR1 genes in plants by methods known in the art such as for example, antisense suppression and co-suppression or sense suppression. Such methods involve the transformation of plants with at least one of the PR1 nucleotide sequences of the invention operably linked to a promoter that drives gene expression in a plant cell. It is recognized that suppressing the expression of an endogenous PR1 gene in a plant can alter the resistance of the plant to plant diseases, particularly diseases caused by plant pathogens.

It is further recognized that with the PR1 nucleotide sequences of the invention, antisense constructions, complementary to at least a portion of the messenger RNA (mRNA) for the PR1 nucleotide sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, for example, transitions and transversions, may be involved.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. It is recognized that the a PR1 protein of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; all of which are herein incorporated by reference.

The expression cassettes and nucleotide constructs comprising the PR1 nucleotide sequences of the invention can be used to transform any plant. In this manner, genetically modified plants, plant cells, plant tissue, seed, and the like can be obtained. Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. AppL. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having inducible expression of the desired phenotypic characteristic identified. Two or more generations may be grown to ensure that inducible or constitutive expression of the desired phenotypic characteristic is stably maintained and inherited and then seeds harvested to ensure expression of the desired phenotypic characteristic has been achieved.

The nucleotide sequences for the PR1 nucleotide sequences of the present invention, and variants and fragments thereof, are useful in the genetic manipulation of any plant when assembled in a nucleotide construct such that the gene sequence is operably linked to a promoter that drives expression of a coding sequence in a plant cell. Such a nucleotide construct can be used with transformation techniques, such as those described herein, to create disease resistance in susceptible plant phenotypes or to enhance disease resistance in resistant plant phenotypes. Accordingly, the invention encompasses methods that are directed to protecting plants against fungal pathogens, viruses, nematodes, insects and the like.

In an embodiment of the invention, methods for enhancing the disease resistance of a plant are provided. The methods involve preparing a nucleotide construct comprising a PR1 nucleotide sequence of the invention operably linked a promoter that drives expression in a plant cell. The methods further involve introducing the nucleotide construct into a plant to enhance the disease resistance of the plant. The methods encompass introducing one, two, three, four, or more PR1 nucleotide sequences into the plant to increase the disease resistance of the plant. Each of such PR1 nucleotide sequences can be operably linked to a promoter that drives expression in a plant. Similarly, one or more of the PR1 nucleotide sequences can be used in a like manner in combination with other known PR1 genes and resistance genes known in the art to increase the resistance of plants to plant diseases.

In one approach, two or more PR1 nucleotide sequences can be introduced into a plant as a single nucleotide molecule. In another approach, each PR1 nucleotide sequence can be introduced a plant on a separate nucleotide molecule. In yet another approach, one or more PR1 nucleotide sequences can be introduced into a first plant and one or more PR1 nucleotide sequences can be introduced into a second plant. The first plant, for example, can be cross pollinated with pollen from the second plant, and progeny resulting therefrom selected for the presence or expression of the desired PR1 nucleotide sequences. Such approaches can also be used to combine in the genome of a plant one or more of the PR1 nucleotide sequence of the invention and other known resistance genes.

Methods for cross pollinating plants are well known to those skilled in the art, and are generally accomplished by allowing the pollen of one plant, the pollen donor, to pollinate a flower of a second plant, the pollen recipient, and then allowing the fertilized eggs in the pollinated flower to mature into seeds. Progeny containing the entire complement of desired heterologous nucleotide sequences of the two parental plants can be selected from all of the progeny by standard methods available in the art as described infra for selecting transformed plants. If necessary, the selected progeny can be used as either the pollen donor or pollen recipient in a subsequent cross pollination.

Transformed plants that express the desired activity can be selected by standard methods available in the art. Such methods include, but are not limited to, determining phenotypes, assaying protein or enzyme activities, immunoblotting using antibodies which bind to the proteins of interest, assaying for the products of a linked reporter or marker gene, PCR amplification of genomic DNA or cDNA to determine the presence of the introduced nucleotide sequences in the genome of the plant and the like.

By “disease resistance” is intended that the disease symptoms that are the outcome of plant-pathogen interactions are avoided or decreased in severity. That is, pathogens are prevented from causing plant diseases and the associated disease symptoms, or alternatively, the disease symptoms caused by the pathogen is minimized or lessened.

By “antipathogenic compositions” is intended that the compositions of the invention have antipathogenic activity and thus are capable of suppressing, controlling, and/or killing the invading pathogenic organism by either directly impacting the invading pathogenic organism or indirectly causing the plant to elicit defense responses which affect the invading pathogenic organism's ability to cause disease symptoms. Antipathogenic compositions of the invention include the PR1 nucleotide sequences of the invention, proteins encoded thereby, and fragments and variants thereof. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

Assays that measure antipathogenic activity are commonly known in the art, as are methods to quantify disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol 34:949-959 and Cammue et al (1992) J. Biol. Chem. 267: 2228-2233, both of which are herein incorporated by reference).

The PR1 proteins of the invention can be used for any application including coating surfaces to target microbes. In this manner, the target microbes include human pathogens or microorganisms. Surfaces that might be coated with the PR1 proteins of the invention include carpets and sterile medical facilities. Polymer bound polypeptides of the invention may be used to coat surfaces. Methods for incorporating compositions with antimicrobial properties into polymers are known in the art. See U.S. Pat. No. 5,847,047, herein incorporated by reference.

Pathogens of the invention include, but are not limited to, viruses or viroids, bacteria, insects, nematodes, fungi, and the like. Viruses include tobacco or cucumber mosaic virus, ringspot virus, necrosis virus, maize dwarf mosaic virus, etc. Specific fungal and viral pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root knot, cyst and lesion nematodes, etc.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug,; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcom maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis, corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murteldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In this manner, the nucleotide sequences for the PR1 nucleotide sequences are provided in expression cassettes as previously described to provide for expression in a plant of interest. Expression of the disclosed PR1 nucleotide sequences may be driven by any promoter that is operable within a plant cell, with the preferred promoter depending upon the desired outcome. Generally, it will be beneficial to regulate expression of the PR1 nucleotide sequence using a constitutive promoter, or an inducible promoter, particularly a pathogen-inducible promoter such as the PR1 inducible promoters disclosed herein. Where expression of a PR1 nucleotide sequences is driven by its native promoter, transformation of a plant with these operably linked sequences results in a plant phenotype exhibiting enhanced disease resistance. Where the transformed plant is the native plant from which the PR1 nucleotide sequences have been isolated, i.e., maize, the transformed plant will exhibit an altered phenotype, or have the operably linked sequences inserted at a new location within its genome. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), the histone H2B promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also U.S. Pat. No. 6,177,611 and WO 99/43797; herein incorporated by reference.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also U.S. Pat. No. 6,177,611, herein incorporated by reference.

Inducible promoters suitable for driving expression of the PR1 nucleotide sequences disclosed herein include, for example, the promoters regulating expression of other pathogenesis-related proteins that are induced following infection by a pathogen; e.g., other PR proteins, SAR proteins, beta-1,3-glucanase, chitinase, etc. See, for example, Redolfi et al (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 1:335-342; Matton et al (1989) Molecular Plant-Microbe Interactions 2:325-331; Somsisch et al. (1986) Proc. Natl. Acad. Sci. USA 83:2427-2430; Somsisch et al. (1988) Molecular and General Genetics 2:93-98; and Yang (1996) Proc. Natl Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiological and Molecular Plant Pathology 41:189-200).

Additionally, as pathogens find entry into plants through wounds or insect damage, a wound-inducible promoter may be used in the nucleotide constructs of the invention. Such wound-inducible promoters or insect-inducible include potato proteinase inhibitor (pin II) gene (Ryan, Ann. Rev. Phytopath. 28:425-449; Duan et al., Nature Biotechnology 14:494-498); wunl and wun2, U.S. Pat. No. 5,428,148; win1 and win2 (Stanford et al. Mol Gen Genet 215:200-208); systemin (McGurl et al. Science 225:1570-1573); WIP1 (Rohmeier et al. Plant Mol. Biol. 22:783-792; Eckelkamp et al. FEBS Letters 323:73-76); MPI gene (Corderok et al. Plant Journal 6(2):141-150); and the like, herein incorporated by reference.

Other inducible promoters include chemical-regulated promoters. Chemical-regulated promoters can be used to modulate the expression of a PR1 nucleotide sequence of the invention in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-i a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced disease resistance within a particular plant tissue. Tissue-preferred promoters include Yamamoto et al. (1997) Plant J. 12(2)255-265; Kawamata et al. (1997) Plant Cell Physiol. 38(7):792-803; Hansen et al. (1997) Mol Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

The expression cassette comprising a desired promoter operably linked to a PR1 nucleotide sequence of the present invention can be used to transform any plant as described elsewhere herein. In this manner, one can obtain genetically modified plants, plant cells, plant tissue, seed, and the like that have enhanced disease resistance to a pathogen.

The PR1 nucleotides sequence of the present invention can be used to transform of any plant species for enhanced disease resistance, including, but not limited to, monocots and dicots. The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants. The following examples are offered by way of illustration and not by way of limitation.

### EXPERIMENTAL

### Example 1

### Isolation of Novel PR1 Nucleotide Sequences from Maize

Six novel PR1 nucleotide sequences were isolated from maize. The PR1 nucleotide sequences of the invention were identified from ESTs based on homology to know PR1 genes and proteins. The new, maize PR1 nucleotide sequences, designated as PR1-6, PR1-7, PR1-8, PR1-9, PR1-10, and PR1-11, are set forth in SEQ ID NOS: 1, 3, 5 7, 9, and 11, respectively. The PR1-7 and PR1-8 nucleotide sequences are full-length cDNA sequences, comprising the entire coding sequences of the respective PR1 proteins. The remaining PR1 sequences of the invention PR1-6, PR1-9, PR1-10, and PR1-11, are partial cDNA sequences and thus comprise only a portion of the coding sequences of the respective PR1 proteins. The amino acid sequences for the polypeptides encoded by the nucleotide sequences designated as PR1-6, PR1-7, PR1-8, PR1-9, PR1-10, and PR1-11 are set forth in SEQ ID NOS: 2, 4, 6, 8, 10, and 12, respectively. The PR1 nucleotide sequences of the invention can be used to isolate their respective, full-length cDNAs and/or genomic clones using standard cloning and DNA isolation techniques known to those of ordinary skill in the art including, but not limited to, screening of maize cDNA or genomic libraries and PCR.

GAP alignments were prepared and used to determine percent identities between each of the PR1 nucleotide and amino acid sequences of the invention and the most closely related sequence in public databases. For nucleotide sequence alignments, only coding regions of the sequences were included in the alignments. The results are indicated below in Table 1.

TABLE 1
Percent Identities of PR1 Nucleotide and Amino Acid Sequences*

| | Nucleotide Sequence | | Amino Acid Sequence | |
|---|---|---|---|---|
| PR1 | SEQ ID NO | % Identity | SEQ ID NO | % Identity |
| PR1-6 | 1 | 61.7 | 2 | 45.5 |
| PR1-7 | 3 | 78.5 | 4 | 52.4 |
| PR1-8 | 5 | 53.1 | 6 | 38.6 |
| PR1-9 | 7 | 61.9 | 8 | 59.7 |
| PR1-10 | 9 | 68.1 | 10 | 57.4 |
| PR1-11 | 11 | 67.9 | 12 | 62.9 |

*Each of the PR1 nucleotide and amino acid sequences of the invention were aligned by the GAP method to the most closely related sequences available in public databases.

### Example 2

### Transformation of Maize with PR1 Nucleotide Sequences by Particle Bombardment and Regeneration of Transgenic Plants

Immature maize embryos from greenhouse donor plants are bombarded with a plasmid containing a PR1 nucleotide sequence of the invention operably linked to a histone H2B promoter and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the PR1 nucleotide sequence of the invention operably linked to a histone H2B promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for enhanced disease resistance.

Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂0 following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 3

### Agrobacterium-mediated Transformation of Maize with PR1 Nucleotide Sequences and Regeneration of Transgenic Plants

For Agrobacterium-mediated transformation of maize with a PR1 nucleotide sequence of the invention, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the PR1 nucleotide sequence of the invention to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated nucleic acid molecule comprising a nucleotide sequence selected from the group consisting of:
a) the nucleotide sequence set forth in SEQ ID NO: 1, 3, 5, 7, 9, or 11;
b) the nucleotide sequence encoding the amino acid sequence set forth in SEQ ID NO: 2, 4, 6, 8, 10, or 12;
c) a nucleotide sequence having at least 80% identity to the coding region of at least one sequence set forth in a), wherein said nucleotide sequence is capable of enhancing the disease resistance of a plant when expressed in the plant;
d) a nucleotide sequence that hybridizes under stringent conditions to at least one sequence that is complementary to a sequence set forth in a), wherein said nucleotide sequence is capable of enhancing the disease resistance of a plant when expressed in the plant; and
e) a nucleotide sequence that is complementary to a sequence of a), b), c), or d).

2. A nucleotide construct comprising a nucleotide sequence of claim 1 operably linked to a promoter that drives expression in a host cell.

3. A vector comprising the nucleotide construct of claim 2.

4. A non-human host cell having stably incorporated in its genome the nucleotide construct of claim 2.

5. A plant cell having stably incorporated in its genome the nucleotide construct of claim 2.

6. A transformed plant comprising in its genome at least one stably incorporated nucleotide construct comprising a PR1 nucleotide sequence operably linked to a promoter that drives expression of a coding sequence in a plant cell, wherein said PR1 nucleotide sequence is selected from the group consisting of:
a) the nucleotide sequence set forth in SEQ ID NO: 1, 3, 5, 7, 9, or 11;
b) the nucleotide sequence encoding the amino acid sequence set forth in SEQ ID NO: 2, 4, 6, 8, 10, or 12;
c) a nucleotide sequence having at least 80% identity to the coding region of at least one sequence set forth in a), wherein said nucleotide sequence is capable of enhancing the disease resistance of a plant when expressed therein;
d) a nucleotide sequence that hybridizes under stringent conditions to at least one sequence that is complementary to a sequence set forth in a), wherein said nucleotide sequence is capable of enhancing the disease resistance of a plant when expressed therein; and
e) a nucleotide sequence that is complementary to a sequence of a), b), c), or d).

7. The plant of claim 6, wherein said plant is a monocot.

8. The plant of claim 7, wherein said monocot is selected from the group consisting of maize, wheat, rice, sorghum, rye, millet, and barley.

9. The plant of claim 6, wherein said plant is a dicot.

10. The plant of claim 9, wherein said dicot is selected from the group consisting of soybean, sunflower, safflower, alfalfa, potato, Brassica spp., cotton, tomato, tobacco, and peanut.

11. The plant of claim 6, wherein said promoter is selected from the group consisting of constitutive, pathogen-inducible, chemical-regulated, wound-inducible, and insect-inducible promoters.

12. Transformed seed of the plant of claim 6.

13. A method for enhancing disease resistance in a plant, said method comprising introducing into at least one cell of said plant at least one nucleotide construct comprising a PR1 nucleotide sequence of claim 1, said PR1 nucleotide sequence operably linked to a promoter that drives expression in a plant cell; wherein the disease resistance of said plant is enhanced.

14. The method of claim 13, wherein said method further comprises regenerating a stably transformed plant from said cell.

15. The method of claim 13, wherein said plant is a monocot.

16. The method of claim 15, wherein said monocot is selected from the group consisting of maize, wheat, rice, sorghum, rye, millet, and barley.

17. The method of claim 13, wherein said plant is a dicot.

18. The method of claim 15, wherein said dicot is selected from the group consisting of soybean, sunflower, safflower, alfalfa, potato, Brassica spp., cotton, tomato, tobacco, and peanut.

19. The method of claim 13, wherein said promoter is selected from the group consisting of constitutive, pathogen-inducible, chemical-regulated, wound-inducible, and insect-inducible promoters.

20. An isolated polypeptide comprising an amino acid sequence selected from the group consisting of:
a) the amino acid sequence set forth in SEQ ID NO: 2, 4, 6, 8, 10, or 12;
b) an amino acid sequence having at least 80% identity to at least one sequence set forth in a), wherein said polypeptide is capable of enhancing the disease resistance of a plant; and
c) the amino acid sequence encoded by the nucleotide sequence set forth in SEQ ID NO: 1, 3, 5, 7, 9, or 11.
